(19) United States
(12) Patent Application Publication
(10) Pub. No.: US 2017/0014315 A1
(43) Pub. Date: Jan. 19, 2017

(54) META-STABLE O/W EMULSIONS

(71) Applicant: BASF SE, Ludwigshafen (DE)

(72) Inventors: Heidi Riedel, Duesseldorf (DE); Thomas Albers, Duesseldorf (DE); Marta Domingo, Barcelona (ES); Bjoern Klotz, Erkrath (DE)

(21) Appl. No.: 15/116,242

(22) PCT Filed: Jan. 23, 2015
(86) PCT No.: PCT/EP2015/051335

§ 371 Date: Aug. 3, 2016

(30) Foreign Application Priority Data
Feb. 5, 2014 (EP) 14153920.5

(51) Int. Cl. A61K 8/06; A61Q 19/00; A61K 8/37; A61Q 17/04; A61K 8/92; A61K 8/31

(52) U.S. Cl. CPC A61K 8/064; A61K 8/062; A61K 8/922; A61K 8/92; A61K 8/31; A61K 8/37; A61Q 17/04; A61Q 19/00; A61K 2800/522; A61K 2800/43; A61K 2800/52

(57) ABSTRACT

Disclosed are processes for the preparation of aqueous meta-stable o/W emulsions with the ability to invert into w/o emulsions when applied to skin. These emulsions are useful for making cosmetic or pharmaceutical compositions, particularly products for skin care.

The present invention relates to the area of producing medium to high viscous, meta-stable o/w emulsions which invert into w/o emulsions when applied to skin. These emulsions are useful for making cosmetic or pharmaceutical compositions, particularly products for skin care.

As water in oil (w/o) emulsion systems are close to the skin's hydrolipid film, they are more effective from the dermatological viewpoint. This emulsion type promotes the long lasting moisturizing efficacy by providing an occlusive film and reinforces the active ingredients action into the stratum corneum. In addition, w/o emulsions leave a lipophilic film on the skin surface which ensures high water repellency, which is an important parameter to maintain high UV protection in sun care applications. Nevertheless, tackiness, combined with greasiness and slow spreading are key factors which tend to decrease cosmetic acceptance, thus counteracting w/o emulsion benefits.

European patent application EP 1174180 B1 (Clariant) claims a process for preparing fine emulsions showing a particle size in the range from 0.1 to 10 μm which are characterized in that a w/o pre-emulsion, comprising at least one w/o emulsifier selected from sorbitol esters, polyglycerol esters, sorbitan esters, fatty acid esters and/or dimethicon copolyols are treated with at least one surfactant in order to invert said w/o fine emulsion into an o/w fine emulsion on condition that the inversion is conducted without increasing the temperature. However, the emulsions thus obtained do not exhibit a sufficient stability, especially when stored at higher temperatures over a couple of days. In addition, the process requires the preparation of an intermediate w/o emulsion which is turned into an o/w emulsion which makes the manufacture time-consuming and little efficient.

Therefore, the complex problem underlying the present invention has been to develop a technology for producing new o/w emulsions exhibiting
high viscosities;
long lasting moisturizing effect;
high water resistance;
enhanced UV protection;
improved active ingredients efficacy;
stability under normal storage conditions;
but inverting into a w/o emulsion when applied to skin.

The present invention is directed on processes of producing aqueous meta-stable o/w emulsions (also called SWOP emulsions) with the ability to invert into w/o emulsions upon application onto the skin.

In a first aspect of the present invention, a process for the preparation of aqueous meta-stable o/w emulsions with the ability to invert into w/o emulsions comprises the following steps:
(i) oil components and w/o emulsifiers are mixed at 10 to 40° C. to form an oil phase (I),
(ii) at least one anionic surfactant and/or non ionic surfactant is mixed at 10 to 40° C. to form an aqueous phase (II),
(iii) oil phase (I) is added to the aqueous phase (II) or vice versa to obtain an o/w emulsion,
(iv) polymeric stabilizers, actives and/or additives are added to the o/w emulsion and
(v) the final emulsion is homogenized.

In a second aspect of the present invention, a process for the preparation of aqueous meta-stable o/w emulsions with the ability to invert into w/o emulsions comprises the following steps:
(i) oil components and w/o emulsifiers are mixed at 50 to 90° C. to form an oil phase (I),
(ii) at least one anionic surfactant and/or non ionic surfactant is mixed at 10 to 40° C. to form an aqueous phase (II),
(iii) oil phase (I) is added to the aqueous phase (II) or vice versa to obtain an o/w emulsion,
(iv) polymeric stabilizers and heat sensitive components are added to the o/w emulsion and
(v) the final emulsion is homogenized.

In a third aspect of the present invention, a process for the preparation of aqueous meta-stable o/w emulsions with the ability to invert into w/o emulsions comprises the following steps:
(i) oil components and w/o emulsifiers are mixed at 50 to 90° C. to form an oil phase (I),
(ii) at least one anionic surfactant and/or non ionic surfactant is mixed at 10 to 40° C. to form an aqueous phase (II),
(iii) oil phase (I) is added to the aqueous phase (II) or vice versa to obtain an o/w emulsion,
(iv) polymeric stabilizers and heat sensitive components are added to the o/w emulsion and
(v) the final emulsion is homogenized.

The emulsions obtained according to the processes of the invention show a viscosity of 3000 to 40000 mPas (Brookfield, 20° C., spindle 5, 10 rpm).

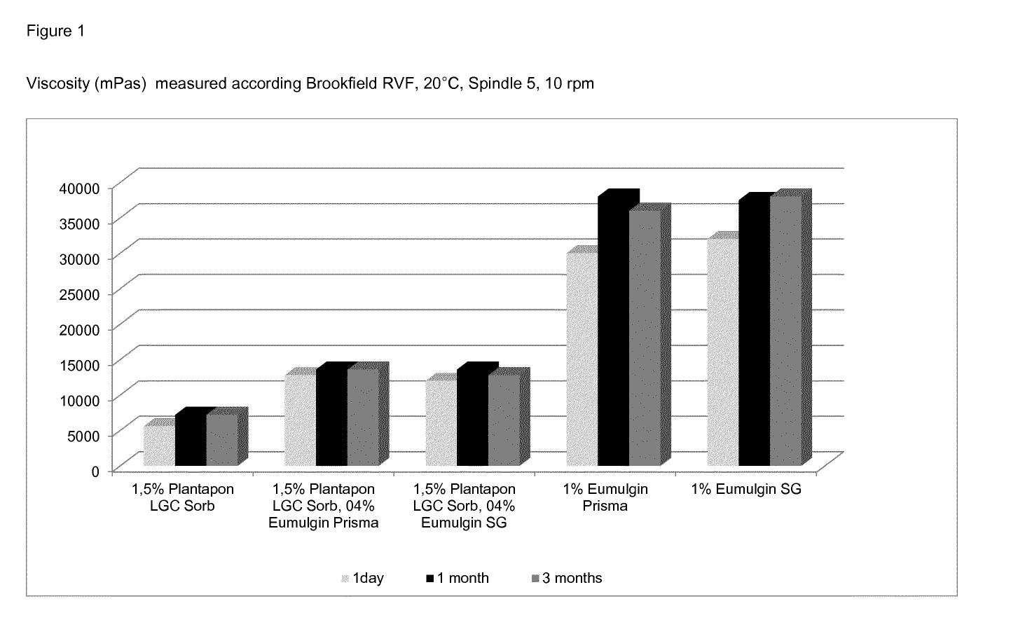

FIG. 1 is a diagram showing the viscosities of emulsions produced according to the invention.

The SWOP emulsions obtained surprisingly show a stronger consistency behavior and a higher richness that is highly appreciated by the consumers.

Surprisingly it has been found that the compositions produced according to the present invention allow the formulation of stable and viscous to high viscous o/w emulsions, having the capacity to invert very quickly into a w/o emulsion while rubbing on the skin. The formation of a lipophilic film on the skin surface is much faster than the normal inversion process that takes place when a standard o/w emulsion is applied and the water phase evaporates.

The aqueous meta-stable o/w emulsions produced according to the invention show a viscosity in the range of 3000 to 200000 mPas (Brookfield, 20° C., spindle 5, 10 rpm). This allows for preparation of highly viscous products for skin care applications. The emulsions produced according to the invention are perfectly suitable for high consistency rich products such as creams. It has been shown that despite of a higher consistency and higher richness the sensoric profile of the SWOP emulsion of the invention remains stable. The high viscosity SWOP emulsion product provides a convenient smooth and soft skin feeling without oily residues on the skin.

In a preferred embodiment of the processes of the invention, at least one anionic and/or non ionic surfactant and optionally at least one oil soluble active and/or additive are added to the oil phase (I).

In a further preferred embodiment of the invention, at least one polymeric stabilizer, water soluble active and/or additive is added to the aqueous phase (II).

In another preferred embodiment of the invention, at least one polymeric stabilizer is added to the oil phase (I).

In the first process of the invention which is the cold/cold process, the temperature is preferably in the range of 15 to 35° C.

In the second process of the present invention which is the warm/cold process, the temperature in step (i) is preferably 70 to 85° C. while the temperature in step (ii) is preferably 15 to 35° C.

In the third process of the invention being the cold/warm process, the temperature in step (i) is preferably preferably 15 to 35° C. while the temperature in step (ii) is preferably 70 to 85° C.

It has been shown that the cold/warm process, the warm/cold process as well as the cold/cold process are highly advantageous with respect to economical aspects such as a low energy input and quick preparation of the emulsion by reducing the time required for warming-up and cooling-down the emulsion. Additionally, the cold/cold process can be performed in a continuous manner enabling a high throughput with higher yields.

Oil Components

Suitable oil components are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkylhydroxy carboxylic acids with linear or branched C₆-C ₂₂-fatty alcohols, in particular dioctyl malate, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonate (Cetiol® CC), Guerbet carbonates, based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.), aliphatic or naphthenic hydrocarbons, such as, for example, squalane, squalene or dialkylcyclohexanes, and/or mineral oils. The preferred oil components, which give the highest benefit from an applicational point of view, can be chosen from the group consisting of tri- or partial glycerides, dialkyl ethers, dialkyl carbonates, liposoluble UV filters like, for example, chemicals filters and/or solutions of pigments, preferably TiO₂ in cosmetic oils, and their mixtures.

Further oil components are hydrocarbons, paraffins, microcrystalline waxes, mineral oils, silicon oils, natural vegetal oils and their mixtures.

The oil component is added in an amount of 8 to 30% by weight based on the emulsion. In a preferred embodiment of the invention, the amount of the oil is 15 to 25% by weight based on the emulsion.

W/O Emulsifiers

Typically, the w/o emulsifiers used in the oil phase are chosen from the group consisting of sorbitan esters and polyglycerol esters:
Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 mol, and preferably 5 to 10 mol, ethylene oxide onto the sorbitan esters mentioned are also suitable.
Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Further typical w/o emulsifiers are polyglycerol esters, polyoxyethylated hydrogenated Castor oil, glycerol ester, polyoxyethylated diester_silicon based w/o emulsifier, polysiloxane-elastomer based w/o emulsifiers and mixtures thereof.

In a preferred embodiment of the invention, polyglycerol esters such as polyglyceryl 2-dipolyhydroxystearate (Dehymuls PGPH) and polyglyceryl-3 diisostearate (Lameform TGI), glycerol ester such as glyceryl oleate (Monomuls 90-O-18) and mixtures thereof are used as w/o emulsifiers. More particularly, polyglyceryl 2-dipolyhydroxystearate (Dehymuls PGPH) is used as an w/o emulsifier.

The w/o emulsifier is used in an amount of 2 to 8% by weight based on the emulsion. In a preferred embodiment of the invention, the amount is from 4 to 6% by weight based on the emulsion.

Surfactants

At least one anionic and/or non-ionic surfactant and water are mixed to form the aqueous phase (II). The surfactant is used in amounts of 0.1 to 10.0% by weight based on the emulsion. Preferred ranges are 0.2 to 2% by weight.

The chemical structure of the surfactant used is critical as it has an impact on the viscosity of emulsion.

The viscosity of the SWOP emulsions produced according to the invention is related to the surfactant(s) used to prepare the emulsions. The use of anionic and/or non-ionic surfactant(s) comprising a carbon chain of 6 to 14 C atoms provides for viscosities in the range of 3000 to 10000 mPas. Emulsions showing a viscosity in that range form flowable liquid products such as fluids and fluid lotions.

Surfactants of a mixture of anionic and/or non-ionic surfactant(s) comprising a carbon chain of 6 to 14 C atoms and anionic and/or non-ionic emulsifier(s) comprising a carbon chain of 15 to 22 carbon atoms gives products having a medium texture quality and showing a viscosity in the range of 10000 to 25000 mPas. This allows for the production of emulsions in the form of lotions including viscous lotions.

Surfactants of anionic or non-ionic emulsifier(s) comprising a carbon chain of 15 to 22 carbon atoms results in products having a high viscosity in the range of 25000 to 200000 mPas. These rich texture high viscosity emulsions are suitable as creams.

The preferred types of anionic surfactants are selected from the group consisting of N-acyl amino acids, in particular acyl glutamates, alk(en)yl oligoglycoside carboxylates, alkyl polyglycolether citrates, esters of sulfosuccinic acid, fatty acids, alk(en)yl sulfates, alk(en)yl sarconisates, alk(en)yl phosphates and their mixtures.
N-Acylamino Acids

Basically, the N-acylamino acids which form component (c1) may be derived from any α-amino acids which can be acylated with fatty acid halides to form N-acylamino acids. Preferred amino acids are glutamic acid, sarcosine, aspartic acid, alanine, valine, leucine, isoleucine, proline, hydroxyproline, lysine, glycine, serine, cystein, cystine, threonine, histidine and salts thereof and, more particularly, glutamic acid, sarcosine, aspartic acid, glycine, lysine and salts thereof. The amino acids may be used in optically pure form or as racemic mixtures. The amino acid components of the N-acylamino acids are preferably derived from glutamic acid and/or aspartic acid, i.e. N-acyl glutamates and N-acyl aspartates are preferably used. In addition, the acyl groups of the N-acylamino acids may be derived from fatty acids corresponding to formula (I):

R¹CO—OH (I)

in which R¹ is a linear or branched acyl group containing 6 to 22 carbon atoms and 0 and/or 1 to 3 double bonds. Typical examples are acyl groups derived from caproic acid, caprylic acid, capric acid, lauric acid, myristic acid, palmitic aid, stearic acid, isostearic acid, oleic acid, elaidic acid, linoleic acid, linolenic acid, gadoleic acid, arachidonic acid, behenic acid and erucic acid and technical mixtures thereof. The N-acylamino acids are preferably derived from technical C₁₂₋₁₈ coconut oil fatty acids. The N-acylamino acids may be present in acidic form, but are generally used in the form of their salts, preferably alkali metal or ammonium salts. The sodium and triethanolamine salts are particularly preferred. Overall, N-cocoyl glutamate is the preferred N-acylamino acid.
Alk(en)yl oligoglycoside carboxylates

The alk(en)yl oligoglycoside carboxylates which can be used in the compositions according to the invention as component (c2) correspond to formula (II):

R²O[G]ₚO[(CH₂)ₘCOO⁻X⁺]ₙ (II)

in which R² is an alkyl or alkenyl radical having from 6 to 22 carbon atoms, G is a sugar unit having 5 or 6 carbon atoms, p is a number from 1 to 10, m and n are numbers from 1 to 5, and X is alkali, alkaline earth, ammonium, alkylammonium, alkanolammonium or glucammonium. The products are obtainable according to the methods known from the art, for example by reaction of alk(en)yl oligoglycosides with halogen carboxylic acids or their salts in alkaline medium in the presence of solvents. The alkyl or alkenyl oligoglycosides carboxylates may be derived from aldoses or ketoses containing 5 or 6 carbon atoms, preferably glucose. Accordingly, the preferred alkyl and/or alkenyl oligoglycoside carboxylates are alkyl or alkenyl oligoglucoside carboxylates. The index p in general formula (II) indicates the degree of oligomerisation (DP degree), i.e. the distribution of mono- and oligoglycosides, and is a number from 1 to 10. Whereas p in a given compound must always be an integer and, above all, may assume a value of 1 to 6, the value p for a certain alkyl oligoglycoside moiety is an analytically determined calculated quantity which is mostly a broken number. Alk(en)yl oligoglycoside carboxylates having an average degree of oligomerisation p of 1.1 to 3.0 are preferably used. Alk(en)yl oligoglycoside carboxylates having a degree of oligomerisation below 1.7 and, more particularly, between 1.2 and 1.4 are preferred from an applicational point of view.

The alkyl or alkenyl radical R² may be derived from primary alcohols containing 4 to 22 carbon atoms, and preferably 8 to 16 carbon atoms. Typical examples are butanol, caproic alcohol, caprylic alcohol, capric alcohol, undecyl alcohol, lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and technical mixtures thereof such as are formed, for example, in the hydrogenation of technical fatty acid methyl esters or in the hydrogenation of aldehydes from Roelen's oxo synthesis. Alkyl oligoglucosides based on hydrogenated C₈-C₁₆ coconut oil alcohol having a DP of 1 to 3 are preferred.

Further on, the alk(en)yl oligoglycoside carboxylates may be derived from carboxylic acids, their salts or esters, in which the acyl moiety comprises 1 to 5, preferably 2 to 4 and more preferably 1 or 2 carbon atoms, while the number of acyl groups in the alk(en)yl oligoglycoside carboxylate may be 1 to 5 and preferably 1 to 3. Moreover, X stands preferably for potassium, ammonium, triethanolammonium and most preferably for sodium. The carboxylic acids, which can be used for preparing the alk(en)yl oligoglycoside carboxylates, usually comprise 1 to 4 carbon atoms; preferably acetic acid, its esters or its salts, particularly its sodium or potassium salt are used. In a preferred embodiment of the present invention, alk(en)yl oligoglycoside carboxylates are used which are obtained by reaction of an aqueous solution of an alk(en)yl oligoglycoside, having e.g. 20 to 70% b.w. solids matter, under nitrogen and in presence of an alkaline catalyst, e.g. alkali hydroxide or alkali carbonate, at a temperature of from 50 to 100° C. with ω-halogen carboxylic acid, its ester or salt, like for example potassium or sodium monocloroacetate in a molar ratio of from 1:0.5 to 1:5, preferably 1:1 to 1:3. The molar ratio of alkali:halogen carboxylic acid, its esters or salts is usually adjusted to 1:0.5 to 1:1.5 and preferably 1:1.1. The preparation of the medium chain C_{12/14} alkyl oligoglycoside carboxylates usually takes place in the absence of organic solvents, while the long chain C_{16/18} alkyl oligoglycoside carboxylates are prepared in the presence of long chain fatty alcohols or 1.2-propylene glycol. These products are obtainable in the market, for example, under the trademark Plantapon® LGC (BASF SE).
Alkylpolyglycolether Citrates

Alk(en)yl polyalkylene glycol ether citrates, which form component (c3), represent mixtures of mono, di and triesters of citric acid and alkoxylated alcohols following formula (III)

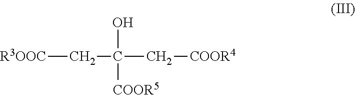

in which R³, R⁴, and R⁵ independently stand for hydrogen or a radical (IV)

R⁶(OCH₂CHR⁷)ₙ (IV)

in which R⁶ is a linear or branched alkyl and/or alkenyl group containing 6 to 22 carbon atoms, R⁷ is hydrogen or methyl, and n is a number from 1 to 20, under the condition that at least R³, R⁴, or R⁵ is different from hydrogen. Typical examples for the alcohol part of the esters are the addition products of on average 1 to 20 moles, and more particularly, 5 to 10 moles of ethylene oxide and/or propylene oxide onto caproic alcohol, caprylic alcohol, 2-ethyl hexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical mixtures thereof. The ethers may have both a conventional homologue distribution and a narrow homologue distribution. It is particularly preferred to use alkyl polyalkylene glycol ethers based on addition products of, on average, 5 to 10, and more particularly of about 7 moles of ethylene oxide with technical C₁₂-C₁₈, preferably C₁₂-C₁₄ fatty alcohol fractions. These products are obtainable in the market, for example, under the trademark Plantapon® LC7 (BASF SE).

The anionic surfactants may also comprise certain amounts of non-ionic emulsifiers or tensides such as alkyl glucosides.

Preferred examples of non-ionic surfactants added are selected from alkylpolyglucosides, polyethoxylated fatty alcohol ethers, polyethoxylated fatty esters, polysorbates, sucrose esters, polyglyceryl esters, pentaerythrityl ester and mixtures thereof.

Examples of Alkylpolyglucosides are Laury Glycoside and Cetearyl Glycoside. Preferred polyethoxylated fatty alcohol ether are steareth-2, steareth-21, ceteareth-6, ceteareth-12, ceteareth-20, Ceteareth-30, beheneth-10, beheneth-25, laureth-7, laureth-23, PPG-1-PEG-9 lauryl glycol ether. Examples of polyethoxylated fatty esters are PEG-100 Stearate, PEG-40 Stearate. Preferred polysorbates are polysorbate 60, polysorbate 40. Sucrose esters are for example sucrose (mono, -or di-, or tri,- or tetra-) stearate. Examples of polyglyceryl esters are PG-10 stearate, polyglyceryl-3-distearate. Pentaerythrityl ester are those having carbon chain length of 6 to 22 atoms.

Particularly preferred examples of anionic surfactants are sodium stearoyl glutamate and disodium cetearyl sulfosuccinate.

In a particularly preferred embodiment of the process of the present invention, acyl glutamate (Plantapon ACG) or alk(en)yl oligoglycoside carboxylate (Plantapon LGC Sorb) in combination with sodium stearoyl glutamate (Eumulgin SG) or disodium cetearyl sulfosuccinate (Eumulgin Prisma) is used in amounts of 1 to 2% and 0.1 to 2%, respectively.

Actives

The nature of the active ingredients (component d) is not critical since the selection fully depends on the required properties of the final product. Typically, the actives are selected from the group consisting of antioxidants, pigments, plant extracts and UV filters (primary and secondary sun protection factors), plant oils, peptids, proteins, amino acids, marine atelocollagen, phytoceramide, photosterols, anti-oxidants, polyphenols, self-tanning agents, UV filters, urea, polyols, hyaluronic acid, sugar or sugar derivatives, sodium PCA and vitamins.

Primary Sun Protection Factors

Primary sun protection factors in the context of the invention are, for example, organic substances (light filters) which are liquid or crystalline at room temperature, and which are capable of absorbing ultraviolet radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor;
4-aminobenzoic acid derivatives, preferably 4-(dimethylamino)benzoic acid-2-ethylhexyl ester, 4-(dimethylamino)-benzoic acid-2-octyl ester and 4-(dimethyl-amino)benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophe-none;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-meth-oxydibenzoyl methane (Parsol® 1789) or 1-phenyl-3-(4′-isopropylphenyl)-propane-1,3-dione and the enamine compounds (BASF). The UV-A and UV-B filters may of course also be used in the form of mixtures. Particularly favourable combinations consist of the derivatives of benzoyl methane, for example 4-tert.butyl-4′-methoxydibenzoyl methane (Parsol® 1789) and 2-cyano-3,3-phenylcinnamic acid-2-ethylhexyl ester (Octocrylene®), in combination with esters of cinnamic acid, preferably 4-methoxycinnamic acid-2-ethylhexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Secondary Sun Protection Factors

Besides the groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides such as D,L-carnosine, D-carnosine, L-carnosine and derivatives thereof (for example anserine), carotinoids, carotenes (for example alpha-carotene, beta-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, alpha-linoleyl, cholesteryl and glyceryl esters thereof) and their salts, dilaurylthiodipropionate, distearyl-thiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages, also (metal) chelators (for example alpha-hydroxyfatty acids, palmitic acid, phytic acid, lactoferrine), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, titanium dioxide (for example dispersions in oils, water or ethanol), zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Iogenic Agents

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prune extract, bambara nut extract, and vitamin complexes.

Specifically, the emulsion produced according to the processes of the invention may contain the following actives:

Moisturizer such as urea, polyols such as glycerol, sorbitol, butylene glycol, propylene glycol, hexanediol. Hyaluronic acid, sugar and sugar derivatives (such as trehalose), sodium PCA.

Vitamins such as water soluble vitamins such as B-vitamins (B1, B2, B6, niacin, pantothen acid, folic acid, biotin and B12) and Vitamin C and derivatives thereof; fat soluble vitamins such as vitamin A, D, E and K and derivatives thereof.

UV filters, plant extracts, plant oils, peptids, proteins, amino acids, marine atelocollagen, phytoceramide, photosterols, anti-oxidants, polyphenols, self-tanning agents such as DHA, AHA/BHA acids, synthetic sarcosine.

Polymeric Stabilizers

Suitable polymeric stabilizers can be selected from the group consisting of polyacrylates, polysaccharides and phyllosilicates.

They are preferably used in concentrations such as 0.05 to 4% based on the emulsion. Polyacrylates and polysaccharides are preferably used in concentrations such as 0.01 to 1%. Phyllosilicates are preferably used in concentrations such as 1 to 4%.

Preferably, the polymeric stabilizers may be selected from the following groups: Hydrokolloids based on polysaccharides such as xanthan, gellan, carboxymethyl cellulose, hydroxypropyl cellulose, methyl cellulose, hydroxypropyl methyl cellulose, hydroxyethyl cellulose, agar-agar, carrageenan, alginates, locust bean gum, guar gum, gum arabic, karaya gum, tragacanth, ghatti gum, pectins and hydroxypropyl guar. Particularily preferred is Xanthan (Rheocare XG).

Polyacrylic acid or_polyacrylates. Particularily preferred are_sodium polyacrylates such as Cosmedia SP, carbomers such as Rheocare C Plus. Most preferred is the combination of sodium polyacrylate (Cosmedia SP) and carbomer (Rheocare C Plus).

Amphipile Polymere such as acrylic acid/C10-30-Alkyl acrylate crosslinked Ccopolymer such as Pemulen® TR1, TR2 Pemulen®, Carbopol® 1382, Carbopol® 1342, Carbopol® ETD 2020;

(meth) acrylic acid/ethyl acrylate/alkyl acrylate copolymer,

crosslinked acrylic acid/vinyl isododecanoate copolymer

acrylic acid/vinylpyrrolidone/lauryl methacrylate terpolymer

Acrylic acid/lauryl (meth) acrylate copolymer,

(meth) acrylic acid/alkyl acrylate/alkyl (POE) allylether,

(meth) acrylic acid/ethyl acrylate/polyoxyethylated lauryl acrylate terpolymer

methacrylic acid/ethyl acrylate/stearyl polyethoxylated terpolymer

methacrylic acid/polyethoxylated nonylphenol acrylate copolymer,

acrylic acid/polyethoxylated stearyl- or cetylmonoitaconate,

copolymers of methacrylic acid, butyl acrylate and hydrophobic monomers having at least one C-chain,

terpolymers of acrylic acid/C15-alkylacrylate/polyethylene glycol acrylate,

salts of partial fatty esters of Copolymers of acrylic acid/dimethylethanolamine,

methacrylic acid/ethyl acrylate/stearyl (polyethoxylated) allyl ethers such as Salcare® SC90 and SC80;

acrylic acid copolymers modified with hydrophobic groups known as HASE polymers (hydrophobic alkali swellable emulsion) comprising acrylates/C10-C30 alkyl methacrylate copolymer such as Luvigel Fit, acrylates/beheneth-25 methacrylate copolymer such as Tinovis GTC.

Most preferred are HASE polymers (hydrophobic alkali swellable emulsion) comprising acrylates/C10-C30 alkyl methacrylate copolymer (Luvigel Fit) sowie acrylates/beheneth-25 methacrylate copolymer (Tinovis GTC).

It has been shown that the polymeric stabilizer displays a remarkable effect on the viscosity built-up of the SWOP emulsion of the invention. Preferably, the use sodium polyacrylate provides viscosities being higher than 3000 Pas. Moreover, the combination of sodium polyacrylate and a carbomer is capable of enhancing the viscosity to values of more than 5000 Pas.

The present invention also refers to the use of said meta-stable o/w emulsions for making cosmetic and/or pharmaceutical compositions, in particular for making skin care compositions, or hair care compositions, or (water-resistant) sun care compositions. Decorative cosmetic articles are also included.

For each specific purpose, the final products may comprise additional ingredients such as additives, like for example additional surfactants, additional oil bodies, additional emulsifiers, superfatting agents, pearlizing waxes, consistency factors, polymers, silicone compounds, waxes, stabilizers, hydrotropes, preservatives, solubilizers, perfume oils, dyes and the like.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Consistency Factors

The consistency factors mainly used are fatty alcohols or hydroxyfatty alcohols containing 12 to 22 carbon atoms, preferably 16 to 18 carbon atoms, and also partial glycerides, fatty acids or hydroxy fatty acids. A combination of these substances with alkyl oligoglucosides and/or fatty acid N-methyl glucamides of the same chain length and/or polyglycerol poly-12-hydroxystearates is preferably used. Further examples are fatty esters of pentaerythritol, hydrogenated vegetable glycerides and mixtures of fatty alcohol sulfates.

Preferred consistency factors are C14 to C22-fatty alcohols (Lanette 14, 16, 18, 22, Lanette O), fatty esters of pentaerythritols (Cutina PES), glyceryl monostearate (Cutina GMS-V), glyceryl mono-/distearate (Cutina MD), hydrogenated vegetable glycerides (Cutina HVG), mixtures of fatty alcohol sulfates specifically sodium cetyl stearyl sulfonate (Lanette E), mixture of cetyl stearyl alcohol and cetyl stearyl sulfonate (Lanette N).

Thickening Agents

Suitable thickeners are polymeric thickeners, such as Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates and electrolytes, such as sodium chloride and ammonium chloride.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxy-substituted carboxylic acids with fatty alcohols containing 6 to 22 carbon atoms, especially long-chain esters of tartaric acid; fatty compounds, such as, for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Silicones

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

Waxes

Besides natural oils used, waxes may also be present in the preparations, more especially natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Stabilizers

Metal salts of fatty acids such as, for example, magnesium, aluminium and/or zinc stearate or ricinoleate may be used as stabilizers.

Hydrotropes

In addition, hydrotropes, for example ethanol, isopropyl alcohol or polyols, may be used to improve flow behaviour. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 Dalton;
technical oligoglycerol mixtures with a degree of self-condensation of 1.5 to 10, such as for example technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Complexing Agents

The complexing agents used may be selected from EDTA, NTA, phosphonic acids, Triton B, turpinal and phenacetin. In addition, reducing agents such as, for example, ascorbic acid, sodium sulfate, sodium thiosulfate and the like may be present. Suitable alkalizing agents are ammonia, monoethanolamines, (L) arginine, AMP, etc.

Perfume Oils

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, □-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of from 0.001 to 0.1% by weight, based on the mixture as a whole.

The total percentage content of auxiliaries and additives may be from 1 to 50% by weight and is preferably from 5 to 40% by weight, based on the particular composition.

The aqueous meta-stable o/w emulsions are used in the following areas of cosmetic applications:
For retarded release of perfume (long lasting perfume effect).
In body care: lotion and crème, for example after sun, self-tan, sun care lotions for women and men, baby care, hand and feet care, products for firming, anti-cellulite products, all-season products, organic cosmetic.
For face care: fluids, serums, lotions, crèmes for sun protection, anti-age/anti-wrinkle applications, day/night/eye care, BB/CC crème, make-up (foundation), organic cosmetic.

Shower-in-Lotion products such as shower crème and shower gel.

### EXAMPLES

### Examples 1 to 10

Meta-stable o/w emulsions (amounts calculated as % b.w.)

| Phase | Compound | 1 | 2 | 3 | 4 | 5 | 6 | 7 | 8 | 9 | 10 |
|---|---|---|---|---|---|---|---|---|---|---|---|
| A | Dehymuls ® PGPH | 4.00 | 4.00 | 4.00 | 4.00 | 4.00 | 4.00 | 4.00 | 4.00 | 4.00 | 4.00 |
| | Polyglyceryl-2 | | | | | | | | | | |
| | Dipolyhydroxystearate | | | | | | | | | | |
| | Isopropyl Myristate | 4.00 | | | | 2.00 | 1.00 | | | 4.00 | |
| | Isopropyl Myristate | | | | | | | | | | |
| | Cetiol C5 | 2.00 | | | 2.00 | | 3.00 | | | 4.00 | 6.00 |
| | Cococaprylate | | | | | | | | | | |
| | Myritol ® 331 | 6.00 | 6.00 | 6.00 | 4.00 | 3.00 | 6.00 | 6.00 | 4.00 | 6.00 | 4.00 |
| | Cocoglycerides | | | | | | | | | | |
| | Cetiol ® CC | 6.00 | 4.00 | 4.00 | 2.00 | 2.00 | | 4.00 | | | |
| | Dicaprylyl Carbonate | | | | | | | | | | |
| | Cetiol AB | | | 6.00 | | 5.00 | 4.00 | | | | |
| | C12-15 Alkyl Benzoate | | | | | | | | | | |
| | Cegesoft ® PFO | | 3.00 | 1.00 | 1.00 | | | | 2.00 | 2.00 | |
| | Passiflora Incarnata | | | | | | | | | | |
| | Seed Oil | | | | | | | | | | |
| | Cetiol ® SB 45 | | 0.50 | | 1.00 | | | 2.00 | 2.00 | | 0.50 |
| | Butyrospermum Parkii | | | | | | | | | | |
| | (EU) | | | | | | | | | | |
| | Cetiol ® J 600 | | | | | 1.00 | | | | 1.00 | |
| | Oleyl Erucate | | | | | | | | | | |
| | CEGESOFT ® PS 6 | | 2.00 | | | 1.00 | | 2.00 | | | 1.50 |
| | Oleyl Erucate | | | | | | | | | | |
| | Dow Corning ® 9040 | | 1.00 | | 3.00 | | | | 1.00 | | |
| | Silicone Elastomer | | | | | | | | | | |
| | Blend | | | | | | | | | | |
| | Cyclopentasiloxane | | | | | | | | | | |
| | (and) Dimethicone | | | | | | | | | | |
| | Crosspolymer | | | | | | | | | | |
| | DC 245 | | 2.50 | | 5.00 | | | | 2.00 | 1.00 | |
| | Cyclomethicone | | | | | | | | | | |
| | Luvitol Light | | | | | | | 4.00 | 6.00 | | 4.00 |
| | Hydrogenated | | | | | | | | | | |
| | Polyisobutene | | | | | | | | | | |
| | Cutina HVG | 1.00 | | | 1.00 | 1.00 | 1.00 | 1.00 | 1.00 | | |
| | Hydrogenated Vegetable | | | | | | | | | | |
| | Glycerides | | | | | | | | | | |
| | Lanette 22 | 0.5 | | | 0.5 | 0.5 | 0.5 | 0.5 | 0.5 | | |
| | Behenyl Alcohol | | | | | | | | | | |
| | Tinosorb S | | | 2.00 | | 1.00 | 2.00 | | | | |
| | Bis-Ethylhexyoxyphenol | | | | | | | | | | |
| | Methoxyphenyl Triazine | | | | | | | | | | |
| | Uvinul MC 80 | | | 4.00 | | 7.00 | 4.00 | | | | |
| | Ethylhexyl | | | | | | | | | | |
| | Methoxycinnamte | | | | | | | | | | |
| | Uvinul A Plus | | | 3.00 | | | 1.00 | | | | |
| | Diethylamino | | | | | | | | | | |
| | Hydroxybenzoyl Hexyl | | | | | | | | | | |
| | Benzoate | | | | | | | | | | |
| | Chrom-Lite Red CI | | | | 1.00 | | | | | 3.00 | |
| | 4506 | | | | | | | | | | |
| | Mica (and) Bismuth | | | | | | | | | | |
| | Oxychloride (and) Iron | | | | | | | | | | |
| | Oxide | | | | | | | | | | |
| | Chinone Crisp Gold | | | | 4.00 | | | | | 5.00 | |
| | S230 V | | | | | | | | | | |
| | Synthetic | | | | | | | | | | |
| | Fluorophlogopite (and) | | | | | | | | | | |
| | Titanium Dioxide | | | | | | | | | | |
| | Eumulgin SG | | | | | 0.2 | | 1.00 | 0.4 | 1.0 | |
| | Sodium Stearoyl | | | | | | | | | | |
| | Glutamate | | | | | | | | | | |
| | Eumulgin Prisma | | | | 0.5 | | 1.00 | | | | |
| | Disodium Cetearyl | | | | | | | | | | |
| | Sulfosuccinate | | | | | | | | | | |
| | Preservative | q.s | q.s | q.s | q.s | q.s | q.s | q.s | q.s | q.s | q.s |
| B | Water | Ad to 100 | | | | | | | | | |
| | Plantapon ACG HC | | | 1.0 | | | | | | | 1.0 |
| | Sodium Cocoyl | | | | | | | | | | |
| | Glutamate | | | | | | | | | | |
| | Plantapon LGC Sorb | 1.5 | 1.5 | | 1.5 | 1.5 | | | 1.5 | | |
| | Sodium Lauryl Glucose | | | | | | | | | | |
| | Carboxylate (and) Lauryl | | | | | | | | | | |
| | Glucoside | | | | | | | | | | |
| | Eumulgin SG | | | | | | | | | 1.0 | |
| | Sodium Stearoyl | | | | | | | | | | |
| | Glutamate | | | | | | | | | | |
| | Eumulgin Prisma | | | | | | | | | | 0.2 |
| | Disodium Cetearyl | | | | | | | | | | |
| | Sulfosuccinate | | | | | | | | | | |
| | NaOH 810%) | q.s. | q.s. | q.s. | q.s. | q.s. | q.s. | q.s. | q.s. | q.s | q.s. |
| | Gycerin | 5.0 | 5.0 | 7.5 | 10.0 | 5.0 | 5.0 | 5.0 | 25.0 | 5.0 | 5.0 |
| | Phenoxyethanol, | 0.5 | 0.5 | 0.5 | 0.5 | 0.5 | 0.5 | 0.5 | 0.5 | 0.5 | 0.5 |
| | Parabene | | | | | | | | | | |
| | Cosmedia SP | 0.8 | | 0.8 | | | 0.5 | | | 0.8 | |
| | Sodium Acrylate | | | | | | | | | | |
| | Rheocare XG | 0.1 | 0.8 | 0.1 | 0.1 | 0.1 | 0.1 | 0.1 | 0.1 | 0.1 | 0.1 |
| | Xanthan Gum | | | | | | | | | | |
| | Rheocare C Plus | | | | | | 0.3 | 0.5 | | | |
| | Carbomer | | | | | | | | | | |
| | Luvigel Fit | | | | 2.0 | | | | | | 1.8 |
| | AcrylatesC/10-30 Alkyl | | | | | | | | | | |
| | Methacrylate Copolymer | | | | | | | | | | |
| | Tinovis GTC | | | | | 2.0 | | | | | |
| | Acrylates/Beheneth-25 | | | | | | | | | | |
| | Methacrylate Copolymer | | | | | | | | | | |
| | Tinovis ADE | | | | | | | | 2.8 | | |
| | Sodium Acrylates | | | | | | | | | | |
| | Copolymer, | | | | | | | | | | |
| | Hydrogenated | | | | | | | | | | |
| | Polydecene, PPG-1 | | | | | | | | | | |
| | Trideceth-6. | | | | | | | | | | |
| | Tinosorb M | | | 5.00 | | | 4.00 | | | | |
| | Methylene Bis- | | | | | | | | | | |
| | Benzotriazolyl | | | | | | | | | | |
| | Tetramethylbutylphenol, | | | | | | | | | | |
| | Aqua, Decyl Glucoside | | | | | | | | | | |
| | (and) Propylene Glycol | | | | | | | | | | |
| | (and) Xanthan Gum | | | | | | | | | | |
| | EDTA | q.s. | q.s. | q.s. | q.s. | q.s. | q.s. | q.s. | q.s. | q.s. | q.s. |
| | Preservative | q.s | q.s | q.s | q.s | q.s | q.s | q.s | q.s | q.s | q.s |
| C | Perfume | 0.3 | 0.3 | 0.3 | 0.3 | 0.3 | 0.3 | 0.3 | 0.3 | 0.3 | 0.3 |
| | Lipofructyl Argan LS | | | 3.00 | | | 3.0 | | | 3.0 | |
| | 9779 | | | | | | | | | | |
| | Argania Spinosa Kernel | | | | | | | | | | |
| | Oil | | | | | | | | | | |
| | Copherol 1250 C | 0.2 | 0.2 | 0.2 | 0.2 | 0.2 | 0.2 | 0.2 | 0.2 | 0.2 | 0.1 |
| | Tocopheryl Acetate | | | | | | | | | | |
| | Ethanol | 5.0 | | | 5.0 | | | | 5.0 | | 2.0 |
| | AMC Advanced | | | 5.0 | | 10.0 | | | | | 3.0 |
| | Moisture Complex NP | | | | | | | | | | |
| | Glycerin (and) Water | | | | | | | | | | |
| | (and) Sodium PCA (and) | | | | | | | | | | |
| | Urea (and) Trehalose | | | | | | | | | | |
| | (and) Hexylene glycol | | | | | | | | | | |
| | (and) Polyquatemium-51 | | | | | | | | | | |
| | (and) Triacetin (and) | | | | | | | | | | |
| | Caprylyl Glycol (and) | | | | | | | | | | |
| | Sodium Hyaluronate | | | | | | | | | | |
| | Litchiderm LS 9704 | | 3.00 | | | | | 4.00 | | | 3.0 |
| | Butylene Glycol (and) | | | | | | | | | | |
| | Litchi Chinensis Pericarp | | | | | | | | | | |
| | Extract | | | | | | | | | | |

The o/w emulsions have been prepared as follows.

1. Preparation of formulations 2 and 3:

Oil components and w/o emulsifiers are mixed at 10 to 40° C. to form an oil phase (I). The anionic surfactants and/or nonionic surfactants, polymeric stabilizers and water are mixed at 10 to 40° C. to form an aqueous phase (II). The oil phase (I) is then added to the aqueous phase (II) or vice versa to obtain an o/w emulsion. The actives and/or additives are added to the o/w emulsion and the final emulsion is homogenized using an Ultra Turrax dispersion unit (4000 r/min).

2. Preparation of formulations 1, 4, 5, 6, 7 and 8:

The oil components and w/o emulsifiers are mixed at 50 to 90° C. to form an oil phase (I). The anionic surfactants and/or non ionic surfactants, polymeric stabilizers and water are mixed at 10 to 40° C. to form an aqueous phase (II). The oil phase (I) is then added to the aqueous phase (II) or vice versa to obtain an o/w emulsion. The emulsion is homogenized using an Ultra Turrax dispersion unit (4000 r/min) and cooled under stirring to 40° C. whereafter the heat sensitive components are added to the o/w emulsion.

3. Preparation of formulations 9 and 10:

The oil components and w/o emulsifiers are mixed at 10 to 40° C. to form an oil phase (I).The anionic surfactants and/or non ionic surfactants, polymeric stabilizers and water are mixed at 50 to 90° C. to form an aqueous phase (II). The oil phase (I) is added to the aqueous phase (II) or vice versa to obtain an o/w emulsion. Subsequently, the emulsion is homogenized using an Ultra Turrax dispersion unit (4000r/min) and cooled under stirring to 40° C. The heat sensitive components are then added to the o/w emulsion.

The o/w emulsions produced according to the processes of the invention, turn into a w/o emulsion very quickly during skin application.

It has been shown that the choice of surfactant is clearly related to the viscosity of the emulsion obtained by the processes of the invention.

FIG. 1 illustrates the impact of the surfactant on the viscosity of the product produced.

The use of 1.5% of Plantapon LCG Sorb (Sodium Lauryl Glucose Carboxylate (and) Lauryl Glucoside) only provides low viscosity emulsions suitable as sprays. The combination of Plantapon LCG Sorb and Eumulgin Prisma (Disodium Cetearyl Sulfosuccinate) results in emulsions of approximately 15000 mPas corresponding to a medium viscosity. These products can be used as body lotions. The same situation applies to the use of Plantapon LCG Sorb and Eumulgin SG (Sodium Stearoyl Glutamate). If Eumulgin Prisma and Eumulgin SG are used alone in correspondingly higher amounts, products with a high viscosity of up to 35000 Pas are obtained that are perfectly suitable as creams.

1. A process for the preparation of aqueous meta-stable o/w emulsions with the ability to invert into w/o emulsions comprising:
(i) oil components and w/o emulsifiers are mixed at 10 to 40° C. to form an oil phase (I),
(ii) at least one anionic surfactant and/or nonionic surfactant and water are mixed at 10 to 40° C. to form an aqueous phase (II),
(iii) oil phase (I) is added to the aqueous phase (II) or vice versa to obtain an o/w emulsion,
(iv) polymeric stabilizers, actives and/or additives are added to the o/w emulsion, and
(v) the final emulsion is homogenized.

2. A process for the preparation of aqueous meta-stable o/w emulsions with the ability to invert into w/o emulsions comprising:
(i) oil components and w/o emulsifiers are mixed at 50 to 90° C. to form an oil phase (I),
(ii) at least one anionic surfactant and/or non ionic surfactant and water are mixed at 10 to 40° C. to form an aqueous phase (II),
(iii) oil phase (I) is added to the aqueous phase (II) or vice versa to obtain an o/w emulsion,
(iv) polymeric stabilizers and heat sensitive components are added to the o/w emulsion, and
(v) the final emulsion is homogenized.

3. A process for the preparation of aqueous meta-stable o/w emulsions with the ability to invert into w/o emulsions comprising:
(i) oil components and w/o emulsifiers are mixed at 10 to 40° C. to form an oil phase (I),
(ii) at least one anionic surfactant and/or non ionic surfactant and water are mixed at 50 to 90° C. to form an aqueous phase (II),
(iii) oil phase (I) is added to the aqueous phase (II) or vice versa to obtain an o/w emulsion,
(iv) polymeric stabilizers and heat sensitive components are added to the o/w emulsion, and
(v) the final emulsion is homogenized.

4. The process according to claim 1, wherein at least one anionic and/or non ionic surfactant and optionally at least one oil soluble active and/or additive are added to the oil phase (I).

5. The process according to claim 1, wherein at least one polymeric stabilizer, water soluble active and/or additive is added to the aqueous phase (II).

6. The process according to claim 1, wherein at least one polymeric stabilizer is added to the oil phase (I).

7. The process according to claim 5 wherein the at least one polymeric stabilizer is dispersed in the aqueous phase (II).

8. The process according to claim 2 wherein the heat sensitive components added are selected from fragrances, actives, emollients and additives.

9. The process according to claim 1 wherein the oil components added are selected from the group consisting of tri- or partial glycerides, dialkyl ethers, dialkyl carbonates, liposoluble UV filters, fatty alcohol ethers, hydrocarbons, paraffins, microcrystalline waxes, mineral oils, silicon oils, natural vegetal oils, and their mixtures.

10. The process according to claim 1 wherein the w/o emulsifiers added are selected from the group consisting of sorbitol esters, polyglycerol esters, polyoxyethylated hydrogenated Castor oil, glycerol esters, polyoxyethylated diesters, silicon based w/o emulsifiers, polysiloxane-elastomer based w/o emulsifiers, and mixtures thereof.

11. The process according to claim 1 wherein the anionic surfactants mixed are selected from the group consisting of acyl glutamates, alk(en)yl oligoglycoside carboxylates, alk(en)yl polyalkyleneglycolether citrates, esters of sulfosuccinic acid, fatty acids, alk(en)ylsulfates, alk(en)yl sarconisates, alk(en)yl phosphates, and their mixtures,
and
the non ionic surfactants added are selected from the group consisting of alkylpolyglucosides, polyethoxylated fatty alcohol ethers, polyethoxylated fatty esters, polysorbates, sucrose esters, polyglyceryl esters, pentaerythrityl esters, and mixtures thereof.

12. The process according to claim 4 wherein the actives added are selected from the group consisting of antioxidants, pigments, plant extracts, plant oils, peptides, proteins, amino acids, marine atelocollagen, phytoceramide, photosterols, anti-oxidants, polyphenols, self-tanning agents, UV filters, urea, polyols, hyaluronic acid, sugar or sugar derivatives, sodium PCA, and vitamins.

13. The process according to claim 1 wherein the polymeric stabilizers added are selected from the group consisting of polyacrylates, polysaccharides, and phyllosilicates.

14. A cosmetic and/or pharmaceutical composition comprising an aqueous meta-stable o/w emulsion produced according to claim 1.

15. The composition of claim 14 wherein the composition is in the form of a skin care composition, a hair care composition, a sun care composition, or a decorative cosmetic article.

16. The process according to claim 2, wherein at least one anionic and/or non ionic surfactant and optionally at least one oil soluble active and/or additive are added to the oil phase (I).

17. The process according to claim 3, wherein at least one anionic and/or non ionic surfactant and optionally at least one oil soluble active and/or additive are added to the oil phase (I).

18. The process according to claim 2, wherein at least one polymeric stabilizer, water soluble active, and/or additive is added to the aqueous phase (II).

19. The process according to claim 3, wherein at least one polymeric stabilizer, water soluble active, and/or additive is added to the aqueous phase (II).

20. The process according to claim 3, wherein the heat sensitive components added are selected from fragrances, actives, emollients, and additives.
